(19) United States
(12) Patent Application Publication
(10) Pub. No.: US 2026/0235622 A1
(43) Pub. Date: Aug. 13, 2026

(54) USE OF GDF-15 AS A NOVEL BIOMARKER

(71) Applicant: Reem MAHRAT, Pleasanton, CA (US)

(72) Inventor: Reem MAHRAT, Pleasanton, CA (US)

(21) Appl. No.: 19/024,024

(22) Filed: Jan. 16, 2025

(51) Int. Cl. G01N 33/68; G16H 10/40

(52) U.S. Cl. CPC G01N 33/6893; G16H 10/40; G01N 2333/475; G01N 2800/362; G01N 2800/7042

(57) ABSTRACT

The present disclosure relates to use of GDF-15 as a novel biomarker for diagnosing, monitoring early-stage cancer, insulin resistance, and autoimmune diseases. Further, the present disclosure also relates to a multipurpose kit and personalized treatment across multiple health domains. Additionally, the present disclosure deals with an AI-driven personalized treatment and point of care (POC) platform using GDF-15 biomarker. There is a disclosure about dual biomarker system comprising GDF 15 and additional biomarker.

### FIELD OF THE INVENTION

The present disclosure generally relates medical diagnostics and personalized healthcare field. The present disclosure pertains to with the use of Growth Differentiation Factor 15(GDF 15 ) as a biomarker for early detection, diagnosis, monitoring, and treatment across a broad spectrum of diseases, including cancer, metabolic disorders, autoimmune diseases, and women's health. It also encompasses the development of a point-of-care (POC) diagnostic platform, such as a lateral flow (LF) assay, for real-time and accessible testing.

### BACKGROUND OF THE INVENTION

Cancer, diabetes, and metabolic disorders such as insulin resistance represent significant public health challenges worldwide. Current diagnostic methods are often limited to late-stage detection or lack sensitivity for early intervention. GDF15, a cytokine belonging to the transforming growth factor-beta (TGF-β) superfamily, has emerged as a promising biomarker due to its involvement in various health conditions, including cancer, diabetes, obesity, autoimmune diseases, and pregnancy-related complications.

Recent studies have demonstrated that elevated levels of GDF15 are associated with metabolic stress, cancer, and early onset of diabetes and insulin resistance. However, to date, there has been no well-defined method for using GDF15 as a comprehensive diagnostic tool for early detection in these diseases. Therefore, there is a need to provide unified diagnostic method using GDF-15 for early detection of various health concerns.

### OBJECTS OF THE INVENTION

An object of the present disclosure is to provide diagnostic method with a unified approach to harnessing GDF15 for life-long, multi-condition monitoring.

Another object of the present disclosure is to provide use of GDF-15 as a novel biomarker for diagnosing early-stage cancer, diabetes, autoimmune, or insulin resistance.

Yet another embodiment of the present disclosure is to provide a kit for diagnosing cancer, diabetes, and insulin resistance.

Another object of the present disclosure is to a point-of-care (POC) diagnostic platform utilizing GDF15.

Yet another object of the present disclosure is to provide an artificial intelligence (AI)-driven personalized treatment based on GDF15 levels.

Another object of the present disclosure is to provide a dual-biomarker diagnostic system to enhance sensitivity and specificity of diagnostics.

And yet another object of the present disclosure is to provide a predictive tool for early-onset menopausal symptoms.

### SUMMARY OF THE INVENTION

The present disclosure relates to a method for assessing biological aging in a subject, comprising:
obtaining a biological sample from the subject, where the sample is selected from the group consisting of blood, urine, saliva, or other bodily fluids;
measuring Growth Differentiation Factor 15(GDF 15 ) levels in the sample;
comparing the measured GDF15 levels to a predetermined baseline indicative of biological age; and
determining the biological age of the subject based on the correlation between GDF15 levels and established biological age markers.

The present disclosure also relates to a method for predicting age-related health outcomes in a subject, comprising:
measuring GDF15 levels in a biological sample, wherein the biological sample is selected from the group consisting of blood or urine, from the subject;
correlating the measured GDF15 levels with the likelihood of developing age-related conditions, wherein the age-related conditions are selected from the group consisting of cardiovascular disease, sarcopenia, osteoporosis, and cognitive decline; and
providing targeted health recommendations based on the assessed risk of age-related conditions.

The present disclosure further relates to a system for managing health outcomes in women at risk of early-onset menopause through GDF15 measurement, comprising:
a database storing baseline and threshold GDF15 levels in both blood and urine, correlated with biological age and menopausal risk;
an algorithm configured to analyze a subject's GDF15 measurement against stored thresholds to assess early-onset menopause risk and biological aging; and
an user interface providing personalized recommendations for health management, including preventative measures, treatment plans, and lifestyle modifications based on GDF15 levels.

The present disclosure additionally relates to a method of using GDF 15 as a biomarker for early prediction, diagnosis, and monitoring of Hyperemesis gravidarum (HG) in pregnant women, comprising the steps of:
obtaining a biological sample from the subject, where the sample is selected from the group consisting of blood, urine, saliva, or other bodily fluids;
measuring Growth Differentiation Factor 15(GDF 15 ) levels in the sample;
comparing the measured GDF15 levels to a predetermined baseline indicative of biological age and stage of pregnancy; and
determining the risk of HG in subject based on the correlation between GDF15 levels and established biological age markers and pregnancy age.

### DETAILED DESCRIPTION OF THE INVENTION

The following is a detailed description of embodiments of the disclosure. The embodiments are in such details as to clearly communicate the disclosure. However, the amount of detail offered is not intended to limit the anticipated variations of embodiments; on the contrary, the intention is to cover all modifications, equivalents, and alternatives falling within the scope of the present disclosures as defined by the appended claims.

The present disclosure relates to a comprehensive diagnostic and therapeutic system that utilizes GDF15 across a variety of conditions and integrates personalized medicine through advanced artificial intelligence (AI) and point-of-care platforms.

Particularly, the present disclosure seeks to fill the gap of lack unified approach for early detection by introducing the use of GDF15 as a diagnostic biomarker to revolutionize early detection and improve patient outcomes.

There is disclosed a novel method for utilizing GDF15 as a biomarker to detect and monitor cancer, weight loss, diabetes, and insulin resistance. This method involves measuring GDF15 levels through non-invasive techniques such as blood assays and establishing a diagnostic reference range to identify elevated GDF15 levels that correlate with early disease onset. The method offers improved accuracy, allowing for early intervention and personalized treatment strategies.

In an embodiment of the present disclosure, the method comprises sample collection, wherein the blood samples are collected from the patient to quantify GDF15 levels. Followed by assay procedure, where GDF15 levels are measured using an enzyme-linked immunosorbent assay (ELISA) or similar immunoassays. Once the assay is performed, there is a need to establish a baseline reference range for GDF15 in healthy individuals. Finally, the method includes the step of comparing patient's GDF15 levels to the established baseline to identify elevated levels indicative of disease.

In yet another embodiment of the present disclosure, the biological sample is urine, enabling a non-invasive approach to biological aging assessment.

In yet another embodiment of the present disclosure, the GDF15 measurement in urine is optimized for stability through controlled sample handling and storage conditions, including temperature and time parameters, to maintain biomarker integrity.

Further, GDF15 levels in urine serve as a non-invasive alternative to blood sampling, allowing routine monitoring of aging biomarkers in settings where blood collection may be impractical.

In another embodiment of the present disclosure, the elevated GDF15 levels serve as a biomarker for certain cancers, particularly lung, prostate, and colorectal cancers. Further, GDF15 is an early indicator of metabolic stress and can predict the onset of type 2 diabetes and insulin resistance. Also, monitoring GDF15 levels helps evaluate metabolic response during weight loss programs and identify abnormalities.

The role of GDF15 as a predictive biomarker for cancer has been validated through clinical studies. Elevated GDF15 levels have been strongly correlated with several malignancies, including lung, prostate, and colorectal cancers. By measuring GDF15 levels in patient samples, the present disclosure provides a method to detect early-stage cancer, allowing for timely treatment, and monitor tumor progression in diagnosed patients.

Additionally, by correlating GDF15 levels with treatment outcomes can help in predicting patient response to therapy. As mentioned above, GDF15 helps in diagnosing insulin resistance by measuring elevated GDF15 levels, enabling early intervention before the development of full-blown diabetes. Further, the present disclosure expands on GDF15's role in metabolic regulation by using it to monitor weight loss programs. Therefore, the elevated GDF15 levels during metabolic stress indicate a poor response to weight loss efforts, helps healthcare providers tailor interventions to individual patients'needs. Hence, through the non-invasive measurement of GDF15 levels, this method offers improved diagnostic accuracy and early intervention opportunities, positioning it as a significant advancement in personalized medicine. Further, GDF15 as a biomarker helps in enhancing patient outcomes by providing healthcare providers with a powerful tool for detecting disease before clinical symptoms appear, thereby reducing mortality rates and improving quality of life. Accordingly, the use of GDF15 as a groundbreaking diagnostic tool across multiple critical health domains, emphasizing its potential to revolutionize early detection and treatment outcomes.

In yet another embodiment of the present disclosure, the GDF-15 has the following essential features:
Multipurpose diagnostic kits that track GDF15 levels over time for comprehensive health monitoring across multiple stages of life.
Point-of-care (POC) platforms, such as lateral flow assays (LF), allowing for real-time and accessible diagnostic testing.
Integration of AI to personalize treatment based on dynamic GDF15 levels, enabling proactive and tailored healthcare solutions.
Dual-biomarker systems combining GDF15 with other biomarkers to enhance diagnostic accuracy for specific conditions, such as cancer and autoimmune diseases.
Therapeutic monitoring, utilizing GDF15 levels to optimize ongoing treatments and prevent disease progression.

Further, GDF 15, or Growth Differentiation Factor 15, is a protein that belongs to the transforming growth factor-beta (TGF-β) superfamily. It is also known as macrophage inhibitory cytokine-1 (MIC-1) and is primarily produced in response to stress or injury. It has an important role in the Body. GDF15 is involved in modulating the inflammatory response. It can act as a protective factor, limiting excessive inflammation and tissue damage. It also plays a significant role in energy metabolism and is associated with changes in metabolic states, such as obesity, diabetes, and cachexia (muscle wasting). Further, GDF15 is implicated in cell growth and division. Therefore, it can promote survival and proliferation in various cell types, including cancer cells. Additionally, the expression of GDF15 is upregulated during cellular stress, such as oxidative stress or tissue injury, serving as a marker of stress and promoting protective mechanisms.

In yet another embodiment of the present disclosure, the elevated levels of GDF15 have been linked to cardiovascular diseases. It may indicate heart stress or damage and is considered a potential biomarker for heart failure and other cardiovascular conditions.

In yet another embodiment of the present disclosure, GDF15 has been shown to offer neuroprotective effects in certain contexts, possibly influencing neurodegenerative diseases.

In yet another embodiment of the present disclosure, GDF15 can promote tumor growth in some cancers, contributing to a more aggressive disease course. It serves as a potential target for cancer therapies.

In another embodiment of the present disclosure, its levels tend to rise with age, and it may be involved in age-related pathologies, making it a point of interest in gerontology research.

In another embodiment of the present disclosure, GDF15 levels in urine serve as a non-invasive alternative to blood sampling, allowing routine monitoring of aging biomarkers in settings where blood collection may be impractical.

In yet another embodiment of the present disclosure, the age-related conditions are selected from the group consisting of cardiovascular disease, sarcopenia, osteoporosis, bone mineral density, Sarcopenia, and cognitive decline.

The present disclosure relates to a method for diagnosing early-stage cancer, diabetes, autoimmune disease, or insulin resistance in a subject, comprising the steps of:
a) collecting a blood sample from a subject;
b) measuring GDF15 levels in a blood sample of the subject;
c) comparing the measured GDF15 levels to a reference range that is established for healthy individuals; and
d) identifying elevated GDF15 levels as indicative of the early-stage cancer, insulin resistance, or metabolic disorder.

Such method is disclosed, wherein the GDF15 levels are measured using an enzyme-linked immunosorbent assay (ELISA) or similar immunoassay technique.

Such method is disclosed, wherein the elevated GDF15 levels are used as a biomarker to detect the presence of cancer, including but not limited to lung, prostate, or colorectal cancer.

Such method is disclosed, wherein the elevated GDF15 levels are indicative of insulin resistance and the early stages of type 2 diabetes.

Such method is disclosed, further comprising monitoring changes in GDF15 levels to assess a patient's response to weight loss interventions and metabolic treatments.

The present disclosure relates to a kit for diagnosing cancer, diabetes, and insulin resistance, comprising:
a) a device for collecting blood samples of a subject;
b) reagents for measuring GDF15 levels through immunoassay; and
c) a reference chart for comparing patient GDF15 levels to establish healthy ranges.

The present disclosure further relates to a diagnostic kit for continuous monitoring of GDF15 levels comprising
a) a diagnostic kit designed for collecting biological samples; and
b) measuring GDF15 levels using immunoassays, wherein the immunoassays is selected from the group consisting of enzyme-linked immunosorbent assays (ELISA) or lateral flow assays (LF).

The present disclosure additionally relates to a point-of-care (POC) diagnostic platform utilizing GDF15 comprising:
a) a lateral flow (LF) assay platform for detecting GDF15 levels in a non-laboratory setting; and
b) a POC platform integrates with mobile applications to upload data directly to healthcare providers.

The present disclosure relates to an artificial intelligence (AI)-driven personalized treatment based on GDF15 levels comprising:
a) integrating AI to analyze real-time GDF15 trends;
b) predicting an onset or progression of diseases; and
c) personalized healthcare recommendations, including dietary interventions, exercise plans, and hormonal therapies, are generated based on continuous GDF15 monitoring.

There is provided a dual-biomarker diagnostic system comprising:
a) a system combining GDF15 biomarker with additional biomarkers to enhance the specificity and sensitivity of diagnostics in autoimmune diseases, cancer, and metabolic disorders.

Further, the present disclosure relates to a predictive tool for early-onset menopausal symptoms comprising:
a) using GDF15 as a predictive marker for identifying women at risk of early-onset menopausal symptoms; and
b) establishing threshold GDF15 levels in pre-menopausal women to predict disease onset well before clinical symptoms manifest.

The invention is further illustrated by the following examples, which is provided to be exemplary of the invention and does not limit the scope of the invention. While the present disclosure has been described in terms of its specific embodiments, certain modifications and equivalents will be apparent to those skilled in the art and are intended within the scope of the present invention.

### EXAMPLES

### Methodology

Sample Collection: Patient blood samples should be collected to measure GDF15 levels.
Assay Procedure: GDF15 levels are quantitatively measured using enzyme-linked immunosorbent assay (ELISA) or other suitable immunoassays.
Patient Population: Studies should include diverse patient populations undergoing weight loss interventions, and those diagnosed with diabetes or showing signs of insulin resistance.
3. Diagnostic Criteria:—Establish a reference range for GDF15 levels in healthy individuals.—Identify elevated GDF15 levels indicative of metabolic syndrome, diabetes, and insulin resistance.
4. Clinical Applications:—Weight Management: Monitor GDF15 levels in individuals undergoing weight loss programs to determine metabolic response.—**Diabetes Monitoring: Utilize GDF15 levels to assess risk of developing diabetes in at-risk populations.
Insulin Sensitivity Assessment: Employ GDF15 as a tool to evaluate changes in insulin sensitivity over time.

To measure GDF15 levels, several specific assays can be employed. Here are some commonly used methodologies:

Enzyme-Linked Immunosorbent Assay (ELISA): A highly sensitive and specific method that uses antibodies to detect and quantify GDF15 in serum or plasma samples. Typically involves the use of a sandwich ELISA format, where capture antibodies bind to GDF15, followed by detection antibodies linked to an enzyme that produces a measurable signal.

Western Blotting: This technique involves separating proteins by gel electrophoresis and transferring them to a membrane, followed by probing with antibodies specific to GDF15. Western blotting allows for assessment of GDF15 protein levels and can also provide information about post-translational modifications.

Luminex xMAP Technology: Utilizes color-coded beads coated with antibodies against GDF15 in a multiplex format, allowing simultaneous measurement of multiple biomarkers. This method combines ELISA-like sensitivity with the efficiency of multiplexing, suitable for high-throughput applications.

Mass Spectrometry:-Advanced technique capable of identifying and quantifying GDF15 at a very high sensitivity level. It is useful for detailed analysis and confirmation of GDF15 among other proteins in complex samples.

Point-of-Care Testing Devices: It is emerging for rapid measurement of GDF15 levels using portable devices. These assays can provide quick results, making them useful in clinical settings for immediate decision-making.

Immunohistochemistry (IHC): While primarily used for tissue samples, IHC can be adapted to visualize GDF15 expression in biopsies, providing localization information along with quantification.

Reverse Transcription Quantitative Polymerase Chain Reaction (RT-qPCR): Although not a direct measurement of protein levels, RT-qPCR can be used to assess GDF15 mRNA expression levels, which correlate with protein production.

### Examples of GDF-15 in Various Diseases

Growth Differentiation Factor 15(GDF-15 ) is a biomarker studied for its diagnostic and prognostic potential in various diseases. Below are some examples:

### 1.Diabetes and Insulin Resistance

In an experiment using human serum from healthy, pre-diabetic, and diabetic patients, GDF-15 levels were measured and analyzed.
Findings:
   GDF-15 levels were lowest in healthy individuals, elevated in pre-diabetic patients, and highest in diabetic patients.
   GDF-15 was also predictive of **insulin resistance**, with higher levels correlating strongly with HOMA-IR scores.
Diagnostic Test Performed: Noted, with thanks.
   ELISA Test: GDF-15 concentrations were quantified using a high-sensitivity enzyme-linked immunosorbent assay.
   Statistical Analysis: ROC curve analysis confirmed GDF-15's ability to differentiate between the groups, with an AUC (Area Under the Curve) exceeding 0.85, indicating high diagnostic accuracy.

### 2. Cardiovascular Diseases

GDF-15 is a well-established biomarker for cardiovascular stress and is associated with adverse outcomes in patients with heart failure or coronary artery disease.

### Findings:

Elevated GDF-15 levels predict major adverse cardiovascular events (MACE) such as heart attack or stroke.
High GDF-15 is correlated with inflammatory pathways and oxidative stress, which are significant in cardiovascular conditions.

### Diagnostic Test Performed:

Immunoassay: Plasma levels of GDF-15 were measured in patients presenting with heart failure.
Validation: Kaplan-Meier survival curves demonstrated that patients with elevated GDF-15 had significantly worse outcomes.

### 3. Cancer

GDF-15 has been studied as a biomarker for several types of cancer, including prostate cancer, colorectal cancer, and pancreatic cancer.

### Findings:

GDF-15 is often overexpressed in tumor microenvironments.
It has been linked to tumor progression, cachexia, and poor prognosis.

### Diagnostic Test Performed:

issue and Blood Analysis: Immunohistochemistry (IHC) and serum ELISA tests were used to measure GDF-15 levels.
Correlation Analysis: Elevated serum GDF-15 was significantly associated with tumor grade and stage.

### 4. Chronic Kidney Disease (CKD)

GDF-15 levels are elevated in CKD and can predict progression to end-stage renal disease (ESRD).

### Findings

High levels of GDF-15 are linked to inflammation and renal fibrosis.
   GDF-15 serves as a prognostic marker for both kidney function decline and cardiovascular mortality in CKD patients.

### Diagnostic Test Performed:

Biochemical Analysis: Serum GDF-15 was measured in CKD patients, with significant differences noted across CKD stages.
Prognostic Model Validation: Combining GDF-15 with eGFR (estimated Glomerular Filtration Rate) improved predictive accuracy.

### 5. Neurodegenerative Disorders

GDF-15 has been explored as a biomarker in Alzheimer's Disease and Parkinson's Disease.

### Findings:

Elevated GDF-15 levels are associated with neuroinflammation and neuronal stress in Alzheimer's patients.
GDF-15 can differentiate patients with mild cognitive impairment (MCI) from healthy controls.
Diagnostic Test Performed:
   CSF and Serum Analysis: GDF-15 levels were measured using multiplex immunoassays.
Longitudinal Study: Elevated GDF-15 levels predicted faster cognitive decline.

### Validation of Diagnostic Ability in the Diabetes Experiment

To evaluate the diagnostic ability of GDF-15 to distinguish between healthy, pre-diabetic, and diabetic patients and predict insulin resistance.

### Methodology:

Sample Group: Serum from 30 healthy individuals, 30 pre-diabetic patients, and 30 diabetic patients.
Test: High-sensitivity ELISA to measure GDF-15 levels.
Metrics: HOMA-IR scores and glucose tolerance tests were performed to validate insulin resistance predictions.

### Results:

GDF-15 showed statistically significant differentiation between the three groups (**p<0.001**).

### ROC Analysis Results:

Healthy vs. Pre-diabetic: AUC=0.88
Pre-diabetic vs. Diabetic: AUC=0.85
Higher GDF-15 levels were strongly correlated with HOMA-IR (r=0.72, p<0.01), confirming its predictive value for insulin resistance.

These examples illustrate GDF-15's versatility as a diagnostic and prognostic biomarker across various diseases, with strong evidence from clinical and biochemical tests supporting its use in differentiating disease states and predicting outcomes.

### ADVANTAGES OF THE PRESENT DISCLOSURE

The present disclosure has the following advantages:
1. Early Detection: The present disclosure allows for the early detection of cancer, diabetes, and insulin resistance through a non-invasive and accurate biomarker.
2. Improved real time Monitoring: GDF15 levels can be tracked over time to monitor patient response to treatment and adjust therapeutic strategies accordingly.
3. Broad Application: The method applies to a variety of diseases, offering a universal diagnostic tool for metabolic and oncological disorders.
4. Personalized Treatment: By identifying elevated GDF15 levels, the method supports precision medicine, tailoring treatments to each patient's unique biochemical profile.
5. Non-invasive, simple blood test.
6. Potentially identifies individuals at risk for developing diabetes and metabolic disorders before clinical symptoms manifest.
7. All-in-One Health Monitoring: The multipurpose diagnostic kit and POC platform offer a convenient and comprehensive solution for lifelong health tracking.
8. Accessible Point-of-Care Testing: The lateral flow assay allows for rapid, real-time diagnostics, bringing healthcare to non-laboratory settings.
9. Personalized Medicine: AI-driven insights provide individualized healthcare solutions, ensuring that treatment plans are tailored to the patient's unique biomarker profile

### INDUSTRIAL APPLICATION

GDF15 (Growth Differentiation Factor 15) can serve as a biomarker for several specific diseases and conditions. Its levels can provide insights into various pathological processes and are associated with the following:
1. Cardiovascular Diseases:—Elevated GDF15 levels are linked to heart failure, myocardial infarction, and other cardiovascular conditions. It is considered a prognostic marker, helping to assess the risk and severity of heart disease.
2. Cancer:—GDF15 can be an indicator of tumor burden and disease progression in various cancers, particularly in gastric, colorectal, and breast cancers. High levels may correlate with poor prognosis and aggressiveness.
3. Metabolic Disorders:—GDF15 is associated with obesity, insulin resistance, and type 2 diabetes. Increased levels are often observed in individuals with metabolic dysfunction, making it a potential marker for these conditions.
4. Chronic Kidney Disease:—Elevated GDF15 levels can indicate renal stress or injury and may correlate with the severity of chronic kidney disease.
5. Neurological Disorders:—GDF15 has been studied as a potential biomarker for neurodegenerative diseases, such as Alzheimer's disease and Parkinson's disease. Increased levels may reflect neuroinflammation or neural stress.
6. Inflammatory Conditions:—Inflammatory diseases, such as rheumatoid arthritis and inflammatory bowel disease, can result in elevated GDF15 levels, reflecting ongoing inflammatory processes.
7. Cachexia:—GDF15 has been implicated in cachexia, a syndrome characterized by weight loss and muscle wasting, often seen in cancer and chronic illnesses.
8. Aging:—GDF15 levels tend to rise with age, and it may serve as a biomarker for age-related health conditions and overall frailty.

The use of GDF15 as a biomarker is still under research, and while it shows promise in indicating disease presence and progression, its application in clinical settings is being further evaluated for specificity and reliability.

Further, the findings of GDF15 biomarker studies provide insights into new therapeutic approaches for aging and menopause, promoting innovations in personalized and preventative healthcare.

### THERAPEUTIC APPLICATIONS

### 1. Precision Treatment Monitoring

Potential: GDF15 reflects treatment response to drugs like metformin, GLP-1 receptor agonists, and immunosuppressants.
Applications:
   Tracking real-time therapeutic effectiveness in metabolic and autoimmune diseases.
   Personalizing medication regimens by tailoring dosages based on GDF15 trends.
Impact: Reduces trial-and-error in treatment and minimizes side effects.

### 2. a Therapeutic Target

Potential: The GDF15-GFRAL axis regulates appetite, energy balance, and immune responses.
Applications:
   Developing appetite-suppressing therapies for obesity and cachexia.
   Modulating immune activity in autoimmune diseases without broad immunosuppression.
Impact: Unlocks novel therapeutic pathways for chronic conditions.

### 3. Adjunctive Role in Clinical Trials

Potential: GDF15 can serve as a surrogate marker in evaluating drug efficacy and safety.
Applications:
   Early-stage clinical trials for therapies targeting metabolic or inflammatory pathways.
   Selecting patients most likely to respond to novel drugs.
Impact: Accelerates the development of innovative therapies and improves trial efficiency.

### 4. Continuous Monitoring in Chronic Diseases

Potential: GDF15 integrates into wearable devices for real-time health tracking.
Applications:
   Monitoring systemic stress and inflammation in chronic diseases.
   Providing personalized behavioral feedback based on dynamic GDF15 data.
Impact: Empowers patients to manage their conditions proactively, reducing hospital visits.

### SPECIFIC EMBODIMENTS OF THE PRESENT DISCLOSURE

The present disclosure relates to a method for assessing biological aging in a subject, comprising:
obtaining a biological sample from the subject, where the sample is selected from the group consisting of blood, urine, saliva, or other bodily fluids;
measuring Growth Differentiation Factor 15(GDF15 ) levels in the sample;
comparing the measured GDF15 levels to a predetermined baseline indicative of biological age; and
determining the biological age of the subject based on the correlation between GDF15 levels and established biological age markers.

Such method is disclosed, wherein the biological sample is urine.

The present disclosure also relates to a method for predicting age-related health outcomes in a subject, comprising:
measuring GDF15 levels in a biological sample, wherein the biological sample is selected from the group consisting of blood or urine, from the subject;
correlating the measured GDF15 levels with the likelihood of developing age-related conditions, wherein the age-related conditions are selected from the group consisting of cardiovascular disease, sarcopenia, osteoporosis, and cognitive decline; and
providing targeted health recommendations based on the assessed risk of age-related conditions.

Such method is disclosed, further comprising a step of validating the clinical correlation between urinary GDF15 levels and blood-based GDF15 levels, establishing predictive equivalency for aging and disease risk across different biological sample types.

The present disclosure relates to a method for predicting early-onset menopausal symptoms in a subject, comprising:
measuring GDF15 levels in a biological sample from the subject;
comparing the measured GDF15 levels to predetermined thresholds associated with risk for early-onset menopause; and
determining the subject's risk of developing early-onset menopausal symptoms based on elevated GDF15 levels.

Such method is disclosed, wherein GDF15 is measured in urine, providing a non-invasive means for early identification of menopausal risk.

The present disclosure relates to a diagnostic kit for assessing biological age and early-onset menopause risk using GDF15, comprising:
a collection device for obtaining a biological sample, wherein the biological sample is selected from the group consisting of blood or urine, from a subject;
an assay configured to detect and quantify GDF15 in the sample; and
instructions for interpreting GDF15 levels in relation to biological age, risk of age-related diseases, and early-onset menopause prediction.

Such diagnostic kit is disclosed, wherein the assay is a highly sensitive enzyme-linked immunosorbent assay (ELISA) or mass spectrometry-based assay optimized for detecting low levels of GDF15 in urine.

The present disclosure further relates to a system for managing health outcomes in women at risk of early-onset menopause through GDF15 measurement, comprising:
a database storing baseline and threshold GDF15 levels in both blood and urine, correlated with biological age and menopausal risk;
an algorithm configured to analyze a subject's GDF15 measurement against stored thresholds to assess early-onset menopause risk and biological aging; and
   an user interface providing personalized recommendations for health management, including preventative measures, treatment plans, and lifestyle modifications based on GDF15 levels.

Such system is disclosed, wherein urine is provided as a non-invasive biomarker sample option, increasing accessibility for regular monitoring in clinical or at-home settings.

The present disclosure additionally relates to a method of using GDF 15 as a biomarker for early prediction, diagnosis, and monitoring of Hyperemesis gravidarum (HG) in pregnant women, comprising the steps of:
obtaining a biological sample from the subject, where the sample is selected from the group consisting of blood, urine, saliva, or other bodily fluids;
   measuring Growth Differentiation Factor 15(GDF15 ) levels in the sample;
   comparing the measured GDF15 levels to a predetermined baseline indicative of biological age and stage of pregnancy; and
   determining the risk of HG in subject based on the correlation between GDF15 levels and established biological age markers and pregnancy age.

Such method is disclosed, wherein the biological sample is urine.

Such method is disclosed, further comprising a step of validating the clinical correlation between urinary GDF15 levels and blood-based GDF15 levels.

The present disclosure also relates to a method for enhancing scientific understanding of GDF15 as a biomarker for aging and menopause comprising the steps of:
utilizing GDF15 as a biomarker to study biological aging processes and early-onset menopausal risk factors;
conducting research on GDF15 levels in urine as a non-invasive alternative to blood for attacking aging and menopause biomarkers; and
analyzing the association between GDF15 levels and health outcomes related to aging and menopause, such as cardiovascular health, bone density, and cognitive function.

The present disclosure further relates to a method for predicting and managing early-onset menopause through GDF15 monitoring in urine, comprising the steps of:
detecting GDF15 levels in a urine sample from a subject;
   identifying elevated GDF15 levels as an early marker of menopausal risk; and
      implementing proactive healthcare interventions tailored to the subject's GDF15 profile, including hormone replacement therapy (HRT), lifestyle modifications, and nutritional support.

1. A method for assessing biological aging in a subject, comprising:
obtaining a biological sample from the subject, where the sample is selected from the group consisting of blood, urine, saliva, or other bodily fluids;
measuring Growth Differentiation Factor 15(GDF15 ) levels in the sample;
comparing the measured GDF15 levels to a predetermined baseline indicative of biological age; and
determining the biological age of the subject based on the correlation between GDF15 levels and established biological age markers.

2. The method of claim 1, wherein the biological sample is urine.

3. A method for predicting age-related health outcomes in a subject, comprising:
measuring GDF15 levels in a biological sample, wherein the biological sample is selected from the group consisting of blood or urine, from the subject;
correlating the measured GDF15 levels with the likelihood of developing age-related conditions, wherein the age-related conditions are selected from the group consisting of cardiovascular disease, sarcopenia, osteoporosis, and cognitive decline; and
providing targeted health recommendations based on the assessed risk of age-related conditions.

4. The method of claim 3, further comprising a step of validating the clinical correlation between urinary GDF15 levels and blood-based GDF15 levels, establishing predictive equivalency for aging and disease risk across different biological sample types.

5. A method for predicting early-onset menopausal symptoms in a subject, comprising:
measuring GDF15 levels in a biological sample from the subject;
comparing the measured GDF15 levels to predetermined thresholds associated with risk for early-onset menopause; and
determining the subject's risk of developing early-onset menopausal symptoms based on elevated GDF15 levels.

6. The method of claim 5, wherein GDF15 is measured in urine, providing a non-invasive means for early identification of menopausal risk.

7. A diagnostic kit for assessing biological age and early-onset menopause risk using GDF15 according to claim 1, comprising:
a collection device for obtaining a biological sample, wherein the biological sample is selected from the group consisting of blood or urine, from a subject;
an assay configured to detect and quantify GDF15 in the sample; and
instructions for interpreting GDF15 levels in relation to biological age, risk of age-related diseases, and early-onset menopause prediction.

8. The diagnostic kit of claim 7, wherein the assay is a highly sensitive enzyme-linked immunosorbent assay (ELISA) or mass spectrometry-based assay optimized for detecting low levels of GDF15 in urine.

9. A system for managing health outcomes in women at risk of early-onset menopause through GDF15 measurement according to claim 5, comprising:
a database storing baseline and threshold GDF15 levels in both blood and urine, correlated with biological age and menopausal risk;
an algorithm configured to analyze a subject's GDF15 measurement against stored thresholds to assess early-onset menopause risk and biological aging; and
an user interface providing personalized recommendations for health management, including preventative measures, treatment plans, and lifestyle modifications based on GDF15 levels.

10. The system of claim 9, wherein urine is provided as a non-invasive biomarker sample option, increasing accessibility for regular monitoring in clinical or at-home settings.

11. A method for enhancing scientific understanding of GDF15 as a biomarker for aging in both men and women and as a biomarker for menopause in women according to claim 1, comprising:
utilizing GDF15 as a biomarker to study biological aging processes and early-onset menopausal risk factors;
conducting research on GDF15 levels in urine as a non-invasive alternative to blood for tracking aging and menopause biomarkers; and
analyzing the association between GDF15 levels and health outcomes related to aging and menopause, such as cardiovascular health, bone density, and cognitive function.

12. A method for predicting and managing early-onset menopause through GDF15 monitoring in urine according to claim 5, comprising the steps of:
detecting GDF15 levels in a urine sample from a subject;
identifying elevated GDF15 levels as an early marker of menopausal risk; and
implementing proactive healthcare interventions tailored to the subject's GDF15 profile, including hormone replacement therapy (HRT), lifestyle modifications, and nutritional support.

13. The method of claim 12, wherein early detection of GDF15 allows for interventions to improve quality of life and manage symptoms associated with early-onset menopause, reducing risks for comorbid conditions such as osteoporosis and cardiovascular disease.

14. A method for validating GDF15 as a urinary biomarker for systemic aging and age-related disease risk according to claim 1, comprising the steps:
establishing the correlation between GDF15 levels in urine and known biological age indicators, including comparisons with blood-based GDF15 measurements;
utilizing sensitive assays to detect low GDF15 concentrations in urine samples; and
determining the predictive value of urinary GDF15 for aging and health outcomes, such as the onset of age-related diseases and functional decline.

15. A method of using GDF 15 as a biomarker for early prediction, diagnosis, and monitoring of Hyperemesis gravidarum (HG) in pregnant women according to claim 1, comprising the steps of:
obtaining a biological sample from the subject, where the sample is selected from the group consisting of blood, urine, saliva, or other bodily fluids;
measuring Growth Differentiation Factor 15(GDF15 ) levels in the sample;
comparing the measured GDF15 levels to a predetermined baseline indicative of biological age and stage of pregnancy; and
determining the risk of HG in subject based on the correlation between GDF15 levels and established biological age markers and pregnancy age.

16. The method of claim 15, wherein the biological sample is urine.

17. The method of claim 15, further comprising a step of validating the clinical correlation between urinary GDF15 levels and blood-based GDF15 levels, establishing predictive equivalency for pregnancy term and HG risk across different biological sample types.

18. A method for enhancing scientific understanding of GDF15 as a biomarker for aging and menopause according to claim 5, comprising the steps of:
utilizing GDF15 as a biomarker to study biological aging processes and early-onset menopausal risk factors;
conducting research on GDF15 levels in urine as a non-invasive alternative to blood for attacking aging and menopause biomarkers; and
analyzing the association between GDF15 levels and health outcomes related to aging and menopause, such as cardiovascular health, bone density, and cognitive function.
